(19) United States
(12) Patent Application Publication
(10) Pub. No.: US 2012/0083634 A1
(43) Pub. Date: Apr. 5, 2012

(54) Adsorbent Regeneration in Light Olefin Recovery Process

(75) Inventor: Jason T. Corradi, Arlington Heights, IL (US)

(73) Assignee: UOP LLC, Des Plaines, IL (US)

(21) Appl. No.: 12/894,825

(22) Filed: Sep. 30, 2010

(51) Int. Cl. C07C 1/20

(52) U.S. Cl. 585/324; 585/639

(57) ABSTRACT

A process is presented for the regeneration of adsorbent beds in the light olefin recovery process. The process uses gas generated in the methanol to olefins process to regenerate adsorbent beds. The gas removes the need for external gases, and the gas can be used in the periodic purging of the methanol to olefins reactor.

### FIELD OF THE INVENTION

The field of the invention relates to the regeneration of adsorbents. In particular, the invention relates to the regeneration of adsorbents in the methanol to olefins process.

### BACKGROUND OF THE INVENTION

The MTO unit includes three separate adsorbent services for the removal of product impurities, primarily water and oxygenates. The adsorbents must undergo periodic, in-situ regeneration. There is insufficient net gas produced to regenerate the adsorbent without importing additional gases. Thus, regeneration requires large quantities of either nitrogen or clean natural gas, which is ultimately vented to the flare or fuel gas header. These external regenerant streams increase utility costs and can potentially introduce new impurities to the process. As an example, an extra purge step is required to remove residual CO2, introduced via natural gas, from the product driers following regeneration.

After the regeneration of the adsorbents, the imported gases are vented to a fuel gas header, or to a flare. The subsequent venting and flaring of these gases is a wasteful expense that can be eliminated.

### BRIEF SUMMARY OF THE INVENTION

A process is presented for the regeneration of adsorbent beds in the methanol to olefins conversion process. The process includes passing an oxygenate stream to a methanol to olefins reactor, wherein an intermediate stream is generated having olefins. The intermediate stream is first quenched to remove water and oxygenates, and then compressed, and separated into a stream comprising heavier hydrocarbons having 4 or more carbons and a lighter stream comprising light olefins. The light olefin stream will have residual oxygenates and CO2 in the stream. The oxygenates and CO2 are removed from the compressed stream, and a recycle oxygenate stream is returned to the methanol to olefins reactor. The compressed light olefins stream is passed through a drying adsorbent bed to remove water that has been generated in the MTO process or the quenching process, before cooling and separating the light olefin products. The dried light olefin stream is passed to a deethanizer to create an overhead stream having ethane, ethylene and lighter components, such as methane, and a bottoms stream comprising primarily propane, propylene and some C4 and heavier hydrocarbons.

The overhead stream is passed to a demethanizer to generate a demethanizer overhead stream and an ethane/ethylene bottoms stream. The demethanizer overhead (DMO) stream is used to regenerate the adsorbent beds used in drying the light olefins stream. The adsorbent beds comprise at least two adsorbent beds where one bed is on-line during operation, and the other adsorbent beds are off-line and regenerated for use when the on-line adsorbent bed is spent. The ethane/ethylene stream is then passed to an ethane/ethylene splitter to recover an ethylene product stream.

The bottoms stream from the deethanizer is passed to a depropanizer where a bottoms stream comprising C4 and higher hydrocarbons is recovered. The depropanizer overhead stream comprises propane and propylene, and is passed to a second adsorbent bed for further removal of residual moisture in the propane/propylene stream. The dried propane/propylene stream is passed to a propane/propylene splitter and the propylene product stream is recovered. The second adsorbent beds comprise at least two adsorbent beds where one bed is on-line during operation, and the other adsorbent beds are off-line and regenerated for use when the on-line adsorbent bed is spent. The DMO stream is passed to the off-line adsorbent beds and the off-line adsorbent beds are regenerated with the moisture removed from the adsorbent beds.

Additional objects, embodiments and details of this invention can be obtained from the following drawing and detailed description of the invention.

### BRIEF DESCRIPTION OF THE DRAWINGS

FIG. 1 is a diagram of the process including the regeneration of the adsorbents; and

FIG. 2 is a specific diagram of one embodiment of the invention.

### DETAILED DESCRIPTION OF THE INVENTION

The methanol to olefin (MTO) process uses three separate adsorbent services. The adsorbents are for the removal of product impurities that include, primarily, water and either residual oxygenates or oxygenates that are generated in the MTO process. One adsorbent unit is for the removal of water from the product stream; a second adsorbent unit is for the removal of CO2 from the methane stream, which is used to regenerate the drier; and a third adsorbent unit is for drying the feed to the olefin cracking unit (OCU) reactor during catalyst regeneration. The adsorbents are periodically regenerated in situ. With the current process there is insufficient net gas produced to regenerate the adsorbents, and therefore, additional gases are imported for use. The regeneration requires large quantities of gas, and therefore large quantities of imported gases are used. The imported gases are either nitrogen or clean natural gas which are expensive, and can include the need to process for the removal of residual sulfur compounds in the gas.

This is an example of a wasteful expense that should be reduced or eliminated. The external regenerant streams increase utility costs and can potentially introduce new impurities to the process. As an example, an extra purge step is required to remove residual CO2, introduced via natural gas, from the product driers following regeneration.

The invention is the use of recycled MTO reactor purge gas for adsorbent regeneration. The total demethanizer overhead satisfies 80% of the total regenerant requirement for all three services combined. The use of recycled purge gas will significantly reduce the need for external regenerant gases. Moreover, it may be possible to eliminate the need for external regenerant gases for certain services by adjusting the adsorbent cycle lengths. Using the demethanizer overhead (DMO) as the sole regenerant for the product driers would eliminate the extra purge step required for residual CO2 removal from the MTO reactor, as the DMO regenerant has only ppb levels of CO2.

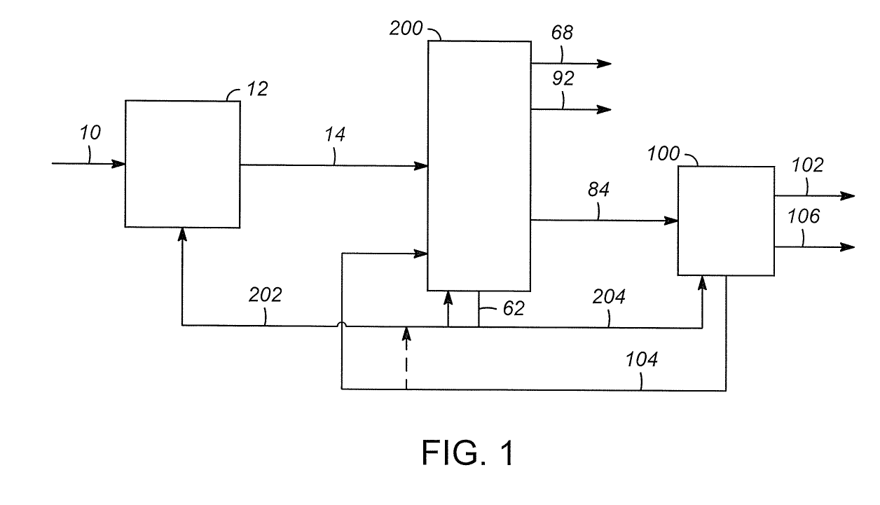

The present invention utilizes a gas stream that is generally vented to the atmosphere, or to a combustion unit. The process is shown in FIG. 1, wherein an MTO reactor generates an intermediate stream comprising light olefins. The intermediate stream is quenched to remove residual oxygenates, and creates a water by-product stream. An oxygenate feedstream 10 is passed to a methanol to olefins reaction zone 12, where an intermediate process stream 14 comprising light olefins is produced. The intermediate process stream 14 is passed to a light olefins separation unit 200. The light olefins separation unit 200 generates an ethylene stream 68, a propylene stream 92, a stream 84 comprising C4+ olefins, and a demethanizer overhead 62. The demethanizer overhead (DMO) stream 62 is passed back to the light olefins recovery unit 200 to regenerate adsorbents in the light olefins recovery process. A portion of the DMO stream 202 can be used to purge the MTO reaction zone 12. Another portion of the DMO stream 204 can be used to regenerate the adsorbent to the reactor feed for the olefin cracking unit 100 during catalyst regeneration. A preferred process uses the DMO stream 62 to regenerate the adsorbent beds in the light olefins separation unit 200 and in the olefin cracking unit, thereby creating an adsorbents off-gas, or spent regenerant, stream. The spent regenerant stream is passed to the MTO reaction unit 12 as a purge gas to purge the MTO reactor. The purge gas returning from the MTO reaction unit 12 is included in the intermediate olefins stream 14 and can be recycled to the adsorbent beds. The loop of cycling the DMO through the adsorbent beds to the MTO reaction unit 12 as a purge gas and back to the adsorbent beds provides sufficient gas without the need for additional gas. The loop includes a means for removing some of the water from the DMO stream as the DMO stream is cycled through the adsorbent beds and the MTO reaction unit 12.

The regenerant gases in the DMO stream 62 can also be used to regenerate guard beds used to protect the catalyst in the MTO reaction zone 12.

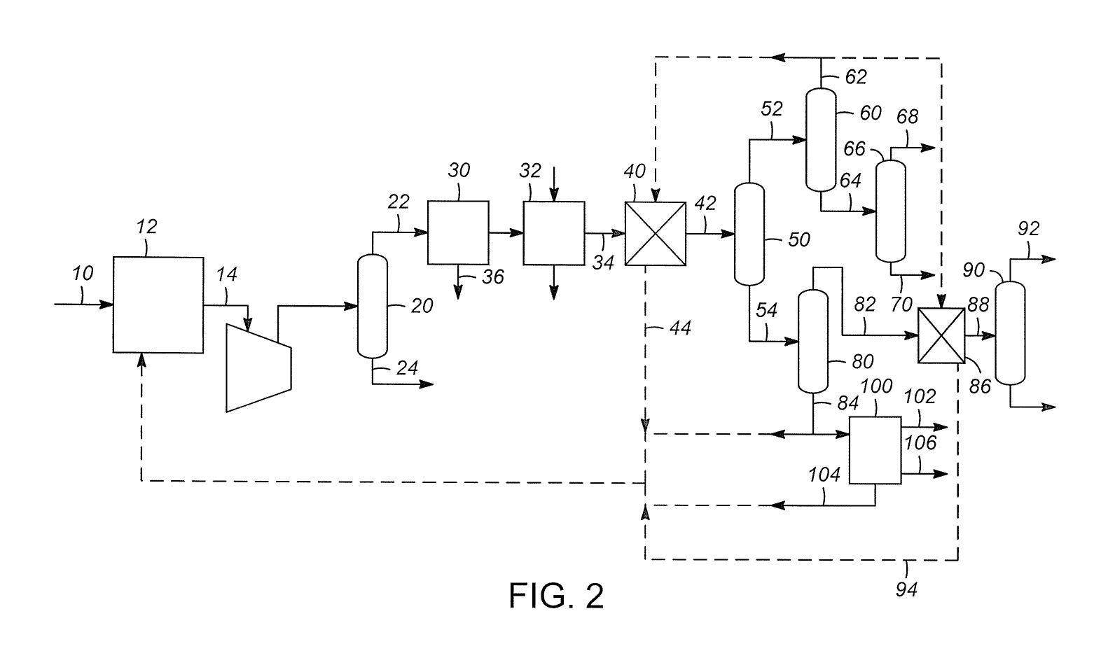

One specific embodiment of the present invention of regenerating adsorbents in a methanol to olefins conversion process is illustrated in FIG. 2. In this embodiment, there are adsorbent beds positioned before the light olefin product splitters, and can also include an adsorbent bed positioned within the olefin cracking unit 100. An oxygenate feedstock 10 is passed to a methanol to olefins reaction zone 12 which generates an intermediate olefins stream 14. The intermediate olefins stream is compressed and passed to a first separation unit 20. The first separation unit can include compressor interstage knockout drums, and creates a first stream 22 comprising light olefins and oxygenates, and a second stream 24 which can comprise C5+ hydrocarbons, dimethyl ether (DME), and heavier oxygenate compounds. The first stream 22 is passed through an oxygenate adsorber 30 and a caustic scrubber 32 to produce a light olefins stream 34 with reduced oxygenate and CO2 content. A recycle oxygenate stream 36 is generated and passed back to the MTO reaction zone 12. The light olefins stream 34 is passed to a first adsorbent bed 40, for removal of water, and other impurities, creating a dry, light olefins stream 42.

The dry light olefins stream 42 is passed to a light olefins recovery unit. The light olefins recovery unit includes a deethanizer 50 which generates a C2 and lighter stream 52 and a C3 and heavier stream 54. The C2 and lighter stream 52 is passed to a demethanizer 60 and creates a demethanizer overhead (DMO) 62 comprising hydrogen and methane. The demethanizer 60 also creates a C2 stream 64 which is passed to an ethane/ethylene splitter 66 creating an ethylene stream 68 and an ethane stream 70. The ethane/ethylene splitter 66 is operated under refrigeration conditions to facilitate the separation, and to reduce the size of the column. The operating conditions for the splitter 66 are such that the ethylene product exits the splitter 66 at a temperature between −20° C. and −35° C., and at a pressure between 1.9 MPa and 2.1 MPa. The feed to the ethylene splitter 66 is cooled and compressed from the demethanizer 60. The DMO is a useful stream for regenerating adsorbents, rather than passing the DMO to a flare, or for disposal with other flue gases. The demethanizer is operated at conditions such that the overhead gases exit the demethanizer at a temperature between 10° C. and 20° C. at a pressure between 700 kPa to 900 kPa.

The first adsorbent bed 40 can comprise a multibed system with one bed on-stream while the other beds are regenerated. When the on-stream bed is saturated, the system is switched such that the on-stream bed is taken off-line, and an off-line bed that has been regenerated is switched to on-stream. At least a portion of the DMO is passed through the off-line first adsorbent beds 40 to regenerate the beds and remove the adsorbed water, creating purge gas streams 44, 94. The purge gas streams 44, 94 are passed to the MTO reaction zone 12 to purge the MTO reactor beds. The purge gas stream exiting the MTO reaction zone 12 is included in the intermediate olefins stream 14 and ultimately recycled to the adsorbent beds 40, 86 as a dry DMO stream for regeneration of the beds. This cycle includes a bleed line for passing excess DMO gas generated as the DMO is cycled through the adsorbent beds 40, 86 and the MTO reaction unit 12.

In one embodiment, the process includes passing the C3 and heavier stream to a depropanizer 80. The depropanizer 80 separates the stream into a propane/propylene stream 82 and a C4+ stream 84. The propane/propylene stream 82 is passed to a second adsorbent bed 86 to dry and remove oxygenates from the propane/propylene stream 82. The purified and dried propane/propylene stream 88 is passed to a propane/propylene splitter 90, where the propylene product stream 92 is recovered. The operating conditions for the splitter 90 are such that the propylene product exits the splitter 90 at a temperature between 40° C. and 60° C., and at a pressure between 2.1 MPa and 2.6 MPa.

The ethane and propane recovered from the ethane/ethylene splitter 66 and the propane/propylene splitter 90 can be either kept as separate streams, or mixed as a combined stream. The ethane and propane products are generally recovered at temperatures between 40° C. and 50° C., and at pressures between 500 and 700 kPa. The C4+, or butane, product stream is generally recovered at temperatures between 40° C. and 50° C., and at pressures between 1.1 MPa and 1.2 MPa.

The second adsorbent bed 86 can comprise a multibed system with one bed on-stream while the other beds are regenerated. When the on-stream bed is saturated, the system is switched such that the on-stream bed is taken off-line, and an off-line bed that has been regenerated is switched to on-stream. At least a portion of the DMO 62 is passed through the off-line second adsorbent beds 86 to regenerate the beds and remove the adsorbed water and other impurities such as oxygenates absorbed by the adsorbent beds 86.

A portion of the DMO stream 62 is passed to the off-line second adsorbent beds 86 to regenerate the adsorbent and removed the water in the adsorbent bed 86.

In an optional arrangement, a portion of the C4+ stream 84 can be mixed with a portion of the DMO stream 62 to regenerate the first and/or second adsorbent beds 40, 86.

In another embodiment, the DMO stream 62 can be used to purge the MTO reactor 12. A portion of the DMO stream 62 is passed to the MTO reactor to purge residual CO2 adsorbed in the reactor. The DMO stream 62 is ideal for this, as residual CO2 has already been removed from the DMO stream, and only very small amount might remain in the DMO stream, or on the order of a few ppm.

In another embodiment, the process can include passing the C4+ stream 84 to an olefin cracking unit 100. The olefin cracking unit 100 cracks the larger olefins, which are separated into a light olefins stream 102, an effluent stream comprising butanes and butylenes 104, and a stream 106 comprising C6 and heavier hydrocarbons. A portion of the butane and butylene stream 104 can be mixed with the DMO stream 62 and passed to the first or second adsorbers 40, 86 during regeneration.

Lower ethylene content is desirable during the purge step of the MTO reactor 12. The mixing of butanes and butylenes can be adjusted to ensure the ethylene content in the DMO stream 62 is sufficiently low so as to prevent any potential coking problems that can arise from a too high level of ethylene in the purge gas. Additionally, the demethanizer 60 can be operated to ensure a sufficiently low amount of ethylene is allowed in the DMO stream 62.

The process can further include separation equipment for separating out heavy materials that are generated by the MTO reactor, prior to compression. An intermediate by-products stream having unreacted materials and heavy materials can be recycled to the MTO reactor, or can be further separated with unreacted oxygenates recycled to the MTO reactor.

The present invention significantly reduces the need for external regenerant or purge gases. By adjusting the regeneration cycles of the adsorbers, the demethanizer overhead can supply the needs without additional external regenerant gases. In addition, this process saves on the clean-up of external regenerant gases. A common regenerant gas is natural gas, and natural gas comes with residual amounts of CO2. These residual amounts of CO2 require treating the natural gas to remove the CO2 prior to using the gas as either a regenerant for the adsorbers, or as a purge gas for the MTO reactor.

While the invention has been described with what are presently considered the preferred embodiments, it is to be understood that the invention is not limited to the disclosed embodiments, but it is intended to cover various modifications and equivalent arrangements included within the scope of the appended claims.

1. A process for regenerating adsorbents in a methanol to olefin process, comprising:
passing an oxygenate stream to a methanol to olefins reactor, thereby generating an intermediate olefins stream;
passing the intermediate olefins stream to an olefins separation unit, wherein the olefins separation unit includes adsorbents for drying and removing contaminants from the olefin streams, and the adsorbents are in fixed beds with the beds not in use in an off-line configuration, and wherein a demethanizer overhead stream is generated;
passing the demethanizer overhead stream to off-line adsorbents to regenerate the off-line adsorbent beds, thereby creating an adsorbent off-gas; and
passing the adsorbent off-gas to the methanol to olefins reactor as a purge gas.

2. The process of claim 1 wherein the adsorbent beds are dryer beds.

3. The process of claim 2 wherein the dryer beds are dryer beds for one of the units selected from the group consisting of the ethylene splitter, the propylene splitter, the olefin cracking unit, and a combination thereof.

4. The process of claim 1 wherein the adsorbent beds are guard beds.

5. A process for regenerating adsorbents in a methanol to olefin process, comprising:
passing an oxygenate stream to a methanol to olefins reactor, thereby generating an intermediate olefins stream;
compressing the intermediate olefins stream, creating a compressed olefins stream;
separating the compressed olefins stream into a first stream comprising light olefins and oxygenates, and a second stream comprising C4 and heavier hydrocarbons;
passing the first stream through an oxygenate adsorber and caustic scrubber, creating a light olefins stream with reduced oxygenate and CO2 content, and a recycle oxygenate stream;
passing the light olefins stream with reduced CO2 content in a first adsorbent bed, creating a dry light olefins stream with reduced water and CO2 content;
separating the dry light olefins with reduced CO2 content stream into a third stream comprising C3 and heavier hydrocarbons and a fourth stream comprising C2 and lighter gases;
separating the fourth stream into a fifth stream comprising ethane and ethylene, and a sixth stream comprising methane and lighter gases;
during regeneration of the first adsorbent bed, regenerating the adsorbent bed with a portion of the sixth stream, thereby creating a seventh stream; and
passing the seventh stream to the methanol to olefins reactor as a purge gas.

6. The process of claim 5 wherein the sixth stream comprises methane and hydrogen.

7. The process of claim 5 further comprising passing the third stream to a depropanizing column, thereby creating an overhead stream comprising propane and propylene, and a bottoms stream comprising C4 and heavier hydrocarbons.

8. The process of claim 7 further comprising mixing a portion of the bottoms stream with the sixth stream, and using the mixed stream to regenerate the first adsorbent bed.

9. The process of claim 7 further comprising:
passing the depropanizing overhead stream through a second adsorbent bed, thereby creating a purified C3 stream; and
passing the purified C3 stream to a propane/propylene splitter, thereby creating a propylene stream and a propane stream.

10. The process of claim 9 further comprising mixing a portion of the bottoms stream with the sixth stream, and using the mixed stream to regenerate the second adsorbent bed.

11. The process of claim 9 further comprising regenerating the second adsorbent bed with a portion of the sixth stream.

12. The process of claim 5 further comprising purging the methanol to olefin reactor with a portion of the sixth stream to remove CO2 adsorbed in the reactor.

13. The process of claim 5 further comprising:
passing the C4 and heavier hydrocarbons stream to an olefin cracking process thereby creating an olefin cracking process effluent;
separating the olefin cracking process effluent thereby creating a stream comprising butanes and butylenes, a stream comprising light olefins and a stream comprising C6 and heavier hydrocarbons; and
passing a portion of the stream comprising butanes and butylenes to the methanol to olefins reactor to purge the reactor.

14. The process of claim 5 further comprising separating the intermediate olefins stream into a byproducts stream comprising unreacted components and the intermediate olefins stream prior to compression.

15. A process for regenerating adsorbents in a methanol to olefin process, comprising:
passing an oxygenate stream to a methanol to olefins reactor, thereby generating an intermediate olefins stream;
compressing the intermediate olefins stream, creating a compressed olefins stream;
separating the compressed olefins stream into a first stream comprising light olefins and oxygenates, and a second stream comprising C4 and heavier hydrocarbons;
passing the first stream in a first adsorbent bed, creating a light olefins stream with reduced oxygenate and CO2 content, and a recycle oxygenate stream;
separating the light olefins stream with reduced CO2 content into a third stream comprising C3 and heavier hydrocarbons and a fourth stream comprising C2 and lighter gases;
separating the fourth stream into a fifth stream comprising ethane and ethylene, and a sixth stream comprising methane and lighter gases;
passing the third stream to a depropanizing column, thereby creating an overhead stream comprising propane and propylene, and a bottoms stream comprising C4 and heavier hydrocarbons;
passing the depropanizing overhead stream through a second adsorbent bed, thereby creating a purified C3 stream;
mixing a portion of the bottoms stream with the sixth stream, and using the mixed stream to regenerate the second adsorbent bed, thereby creating a seventh stream;
during regeneration of the first adsorbent bed, regenerating the adsorbent bed with a portion of the sixth stream, thereby creating an eighth stream; and
passing the seventh and eighth streams to the methanol to olefins reactor as a purge gas.

16. The process of claim 15 further comprising:
passing the C4 and heavier hydrocarbons stream to an olefin cracking process thereby creating an olefin cracking process effluent;
separating the olefin cracking process effluent thereby creating a stream comprising butanes and butylenes, a stream comprising light olefins and a stream comprising C6 and heavier hydrocarbons; and
passing a portion of the stream comprising butanes and butylenes to the methanol to olefins reactor to purge the reactor.

17. The process of claim 16 further comprising passing the purified C3 stream to a propane/propylene splitter, thereby creating a propylene stream and a propane stream.
